(19) United States
(12) Patent Application Publication
(10) Pub. No.: US 2004/0255360 A1
(43) Pub. Date: Dec. 23, 2004

(54) Central line access and support garment

(76) Inventor: Mary C. Bass, Earlysville, VA (US)

Correspondence Address: Mark J. Young, Suite 227, 9951 Atlantic Blvd., Jacksonville, FL, 32225, US

(21) Appl. No.: 10/897,777

(22) Filed: Jul. 22, 2004

Related U.S. Application Data
(63) Continuation-in-part of application No. 10/425,316, filed on Apr. 29, 2003.

(51) Int. Cl. A61M 37/00

(52) U.S. Cl. 2/69

(57) ABSTRACT

A central line access and support garment provides an effectively tight inner layer to support and protect central line port sites and external lines, without applying excessive pressure that would cause discomfort. The garment protects a multitude of port site locations. Releasable shoulder closures allow for easy access to the port sites and lines while providing an easy to wear garment for the patient. An inner layer is tight enough to protect the port site, securely holding the bandage, if used at the port site, against the patient's body. The garment offers the flexibility of placing the external lines anywhere on the patient's upper torso for comfort to the patient. An elastic band around the central body cavity serves as a protective shelf to keep external lines from hanging freely and thus pulling on the port site. An outer layer serves to protect the inner layer, absorbing stresses caused by outer garment movement and providing an additional layer of protection for the port site and external lines.

### RELATED APPLICATIONS

This application claims priority to U.S. Non-Provisional application Ser. No. 10/425,316, filed Apr. 29, 2003, the entire contents of which are hereby incorporated by reference herein.

### FIELD OF INVENTION

The invention relates to the field of medicine. More particularly, this invention relates to garments for providing access and support to central line catheters for safety, maintenance, comfort, and decreased exposure of an implantation site.

### BACKGROUND

A central line catheter is a surgically implanted line having one end threaded into a vein. The other end, which remains outside the body, provides access for administering medication, performing blood transfusions and intravenous infusions and taking blood for tests. Catheters may remain in place for a varied length of time and possibly permanently. A small cuff around the central line is typically located under the skin just above the exit or port site. Scar tissue under the skin eventually grows around the cuff to hold the line in place. Until this happens a stitch may hold the line in place. A bandage may also be used as a temporary or daily dressing to protect the port site from infection, damage to the site, and premature or accidental removal of the catheter. A port (i.e., access port) at the end of the external portion of the catheter line enables access for injections and desired procedures. A catheter may contain a plurality of lines (lumens) where multiple fluids will be infused. In such case, an access port is provided at the end of each lumen.

It is important to secure the port or line site in patients with central lines. These sites require daily maintenance for the proper functioning of the catheter. Doctors have generally used gauze pads, various forms of adhesives, plastic shields, and garments, such as jog bras, to secure the catheter to the patient. Without such security, the patient runs the risk of infection, snagging of the line and causing damage and/or accidental or unintentional removal of the line prematurely, and increased scarring due to the movement of the catheter. It is especially important during the first few weeks after the implantation of the catheter that it be held securely to the body of the patient to allow for the scar tissue to form on the catheter cuff which ensures long term security and use of the catheter device.

Although during the day many women can use bras and men can use necklaces with clamps to hold the catheter in place, comfort and safety during sleeping has continually posed problems. Necklaces tend to shift with the patient during the night, and wearing a bra at night is not comfortable. Neither of these options offers any security to the port site or external catheter lines when in a reclined mode, such as sleeping, thus posing a threat to damaging the site, premature or accidental removal of the catheter, and discomfort from rolling over onto the ends (i.e., access ports) of the external lines.

Patients have been encouraged to purchase sports bras and other women's accessories as a method for keeping the catheter line secure and the site protected. The negatives of these alternatives are many. For example, a large male will not find even the largest sports bra comfortable. A female, with a sports bra fitted to her size, can also find these very uncomfortable as they cross over the neck area in a sensitive place where the internal catheter is close to the skin. Sports bras are extremely tight against the body as a whole. The sports bra design, although very secure, often crosses over the clavicular area of the patient, the site where most central lines cross into the neck. This not only causes discomfort around the mid-section of the patient, but also in the neck area which is often affected by the central line placement. A sports bra is also very difficult to take on and off. This would even be more difficult for a patient with limited range of upper body motion or upper body weakness.

T-shirts, a few sizes too small, are also used to keep the line tight against the body of the patient and the dressing on the implantation site secure. However, as with sports bras, getting these garments on and off can be difficult for patients with a limited range of upper body motion. Such garments also do not facilitate access to the implant area or the lines themselves. Furthermore, standard t-shirts lack a containment and support means for keeping the external catheter lines above the midsection. Thus, increased tension can be placed on the port site due to the lack of support for the weight of the external lines.

Shoulder straps, spaghetti straps, or thinner shoulder pieces have the disadvantage of either not covering the port site or irritating the port site by either rubbing it or getting under the bandage designed to protect the site. If the port site is located centrally on the patient's chest, these types of garments might not even cover the port site thus serving no purpose other than holding the external catheter lines above the midsection.

Other garments addressing this issue have been designed to assist in the external care of the catheter lines. For example, Intravenous Catheter Holder, Schneider, U.S. Pat. No. 4,578,062 (Mar. 25, 1986), describes a pouch to contain the catheter and access through the shoulders for maintaining the site. Another example, Garment for Concealing Patient Medical Appliances, Ojoyeyi, U.S. Pat. No. 6,477,710 B1 (Nov. 12, 2002) describes several pockets to hold catheter lines, however, it does not offer the easy shoulder access as the garment described in U.S. Pat. No. 4,578,062 (Mar. 25, 1986). The pouch and pocket both offer an area to store the line versus having it hang free or attached to clothing or a necklace by a clip. However, there is no option as to the placement of the pouch or pocket and, thus, this can be uncomfortable to some patients. The clips can be cold and hard, the necklaces can hang around the sensitive areas of the neck. The pouches and pockets do not offer any options in the placement of the lines against the body, therefore causing discomfort when hard clamps and access ports are pressed against the body.

Thus, an improved garment is needed to secure the line and implantation site, provide comfort to the patient, accessibility to medical staff, ease in cleaning the port site, and ease in putting on and taking off the garment.

### SUMMARY

A garment in accordance with an exemplary embodiment of the present invention includes an inner layer of fabric relatively tightly fitted to the body to secure the dressing to a port site. The garment includes an inner shelf lining of elastic to allow the patient flexibility in locating the end(s) of the catheter in the garment, but keeping the end(s) from falling low enough to pull at the implantation site. In a preferred embodiment, an outer layer is attached to the inner layer, such as at the shoulders, arms and neck, to provide comfort and to reduce stress on the inner layer, lines and port site from movement of outer garments. Also in a preferred embodiment, releasable closures are provided at the shoulders to enable access for easy cleaning, treatment procedures, and putting on and taking off the garment. The neck is preferably high enough to protect any port site a doctor may choose when inserting a catheter into the patient, but low enough not to press on the clavicular area. The inner garment and elastic band are preferably tight enough to secure the port site, dressing, and lines, but also provide the patient with options of locating the end(s) of the catheter line where it is most comfortable for that patient.

It is, therefore, an object of the present invention to provide a garment that will secure central catheter lines or other similar devices, including the external portion of lines and port sites.

It is another object of the present invention to provide a garment that facilitates access by medical staff and care givers for cleaning and treating, and placing on and removing the garment, especially for patients with limited range of upper body motion.

It is also another object of the present invention to provide a garment that allows a patient to choose where a catheter line is held against the body.

It is yet another object of the present invention to provide a garment that accommodates a range of port sites, thereby enhancing doctors' flexibility in site selection.

It is another object of the present invention to provide a garment that is comfortable to a patient in a reclined position, such as sleeping, and enables the patient to change positions while reclined without damaging the catheter, prematurely or accidentally removing it, or incurring discomfort. For example, patients can comfortably sleep in any position.

It is a further object of the present invention to provide a garment that protects catheter lines and a port site while providing security of the line and comfort to the patient.

### BRIEF DESCRIPTION OF THE DRAWINGS

The foregoing and other objects, features and advantages of the present invention will become better understood with reference to the following description, appended claims, and accompanying drawings where:

FIG. 1 conceptually depicts the front of an outer layer of a garment in accordance with an exemplary embodiment of the present invention. The outer layer is designed as a sleeveless t-shirt with releasable closures at the shoulders. The closures are preferably adjustable to allow for a customized fit of the patient. The length is preferably long enough to tuck into pants, skirts, pajama bottoms, shorts, or any other lower body garment.

FIG. 2 conceptually depicts the back of an outer layer of the garment in accordance with an exemplary embodiment of the present invention. The outer layer design is the same as in FIG. 1.

FIG. 3 conceptually illustrates the front of an inner layer of a garment in accordance with an exemplary embodiment of the present invention. The inner layer is preferably secured to the outer layer at the neck, shoulder and arm seams. The shoulder seams are preferably adjustable for a customized fit. The material is a relatively tight, stretchable fabric, tighter in fit than the outer layer, providing security to the port site. An elastic body band is attached to the inner layer and sits low enough on the patient for comfort yet high enough to inhibit the catheter lines from falling downward and pulling on the port site.

FIG. 4 conceptually illustrates the back of an inner layer of the garment in accordance with an exemplary embodiment of the present invention.

FIG. 5 conceptually illustrates the front of a garment in accordance with an exemplary embodiment of the present invention along with a variety of possible placements (each denoted with an X), but not limited to these shown, for a catheter port, demonstrating versatility of the garment. The garment is low enough not to interfere with clavicular areas, yet high enough to provide security to a wide variety of port sites.

FIG. 6 conceptually illustrates the front of a garment in accordance with an exemplary embodiment of the present invention along with various positions of a catheter line (each position being represented by a curved line ending with an arrow) to provide comfort to a patient. A garment in accordance with the present invention permits a patient to place the lines under an arm, between breasts, or along the chest area, or to separate them to avoid rubbing, pressure points, and discomfort.

FIG. 7 conceptually illustrates releasable closures for the shoulder regions of an outer layer of a garment in accordance with an exemplary embodiment of the present invention.

FIGS. 8, 8A and 8B conceptually illustrate dimensions for standard sizes of an inner layer and an outer layer of a garment in accordance with an exemplary embodiment of the present invention.

FIGS. 9 and 10 illustrate a sports bra and garment with spaghetti straps and particular areas of conflict or discomfort with a catheter port or line.

### DETAILED DESCRIPTION

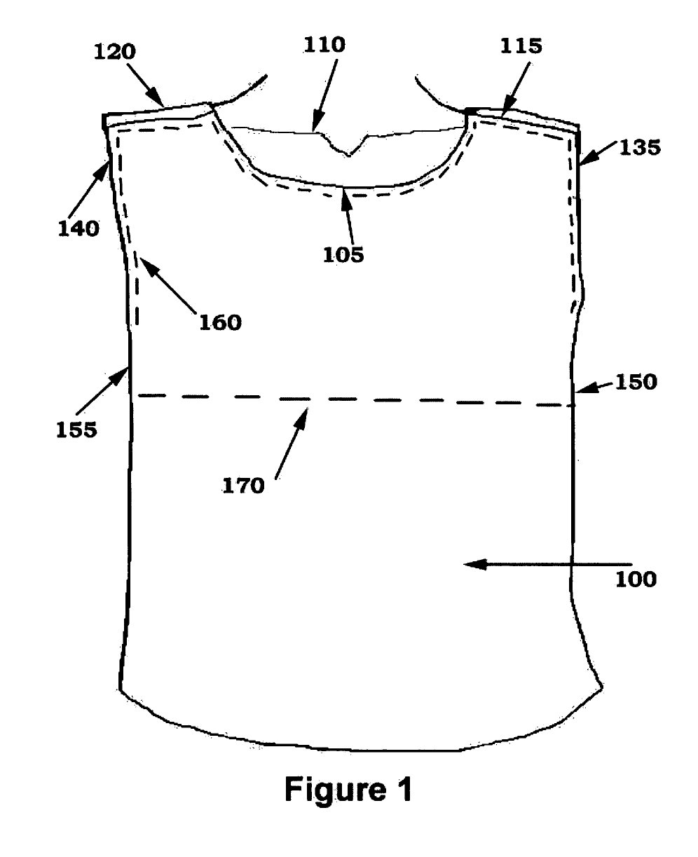

Referring now to FIG. 1, a front view of an outer layer 100 of a central line access and support garment in accordance with an exemplary embodiment of the present invention is shown. The outer layer 100 comprised of material such as cotton, a stretchable synthetic fiber material (e.g., Lycra® by E. I. du Pont de Nemours and Company), jersey knit, or other fabric, provides an undershirt garment for appearance. It also protects an inner layer (discussed below) by reducing or eliminating frictional motion from movement of outer clothing worn by a patient. The outer layer 100 preferably has a neck line 105 just below the patient's clavicular region 110 to avoid contact with a catheter line which may cross into the neck region. The shoulders 115 and 120 of the outer layer 100 are preferably broad enough to provide security without being uncomfortably tight.

The outer layer 100 is preferably comprised of a front panel and a back panel, discussed below, as in a conventional manner for shirts. The front panel and back panel may be joined by stitching along sides 150 and 155 in a conventional manner.

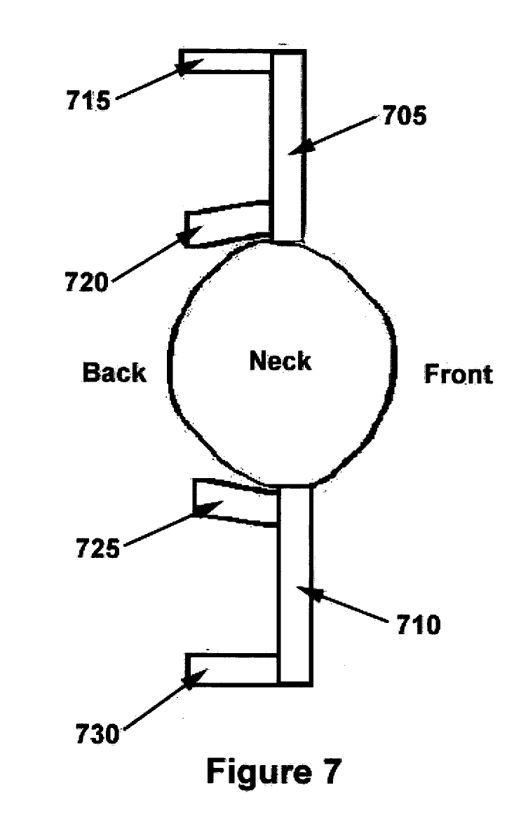

Left and right shoulder portions or sections (115 and 120, respectively), each having front panel and back panel portions (or sections), are provided. The shoulder portions 115 and 120 of the front panel are preferably releasably joined to the shoulder portions of the back panel using releasable closure means, such as hook and loop fasteners (e.g., Velcro® hook and loop fasteners by Velcro Industries B.V.); though other releasable closure means (e.g., buttons, snaps and zippers) may be used without departing from the scope of the present invention. In an exemplary embodiment, snag free releasable closures such as Snag-Free Velcro® fasteners with buffers are used to eliminate snagging of adjacent fabrics and to resist lint build-up. The releasable closure means may allow adjustment of the shoulder portions to accommodate a range of patient physiques and preferences. By way of example and not limitation, the shoulder portions 115 and 120 of the front panel of the outer layer may have loop portions of a hook and loop closure 705 and 710, as shown in FIG. 7, horizontally across the width of the shoulder seams 115 and 120. The back panel may have cooperating hook portions of hook and loop closures 715, 720, 725, 730 vertically along the sides of the shoulder strap to allow for customized adjustment.

In use, a patient may open the releasable closures 715-730 at the shoulder sections to facilitate removing and placing on the garment. Additionally, a patient may open the releasable closures for one or both shoulders to facilitate access to the catheter line and/or port site.

The outer garment preferably provides arm holes 135 and 140 large enough to comfortably accommodate a patient's arms, but small enough to provide security for the catheter. Adjustment of the releasable closure means 715-730, preferably adjusts the sizes of the arm holes, allowing the garment to accommodate various physiques and preferences.

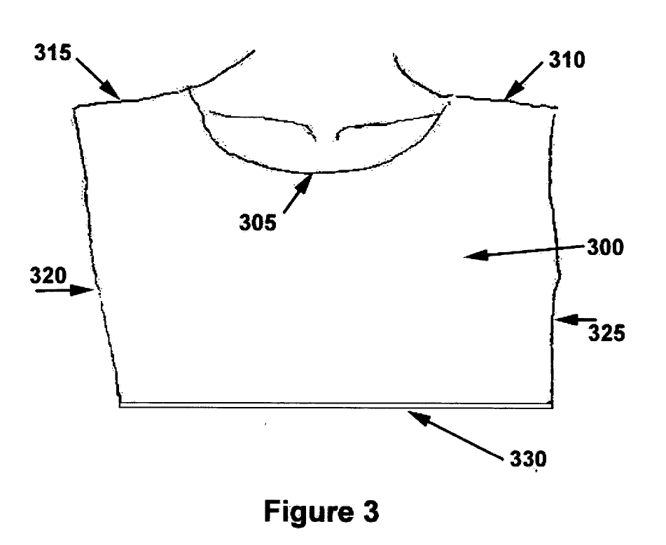

The outer layer 100 is joined to the inner layer 300 shown in FIG. 3, along the arm holes 135 and 140, the shoulders 115 and 120, and the neckline 105 as indicated by the dotted line 160. The inner layer 300 is shorter in length than the outer layer 100 as indicated by the dotted line 170.

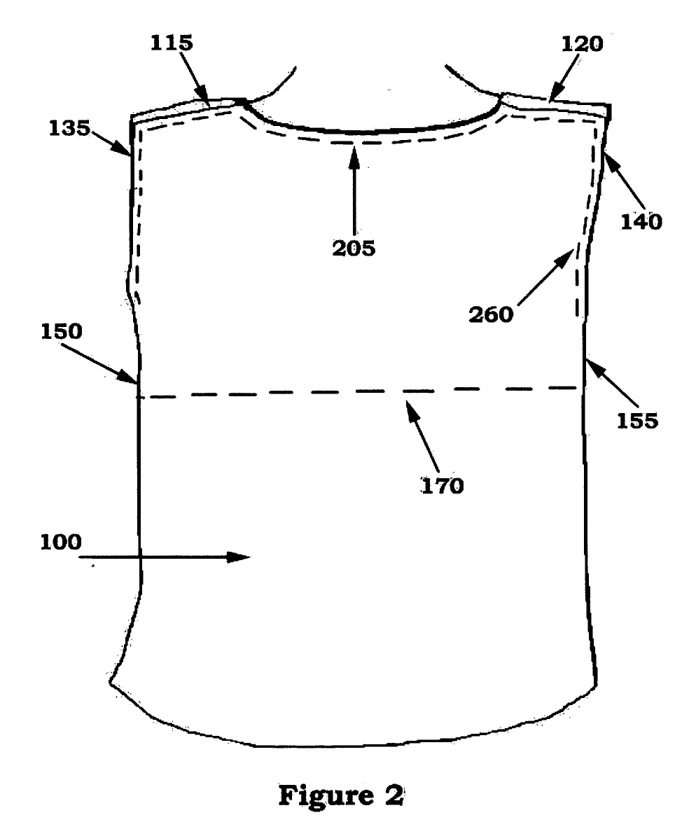

In FIG. 2, a rear view of the outer layer 100 of the central line access and support garment in accordance with an exemplary embodiment of the present invention is shown. The outer layer 100 remains the same as described in FIG. 1. The neckline 205 is closer to the base of the neck of the patient to provide enhanced fit, comfort, and security. Arm holes 135 and 140 remain the same as described in FIG. 1. The shoulder closures 115 and 120 remain the same as described in FIG. 1, and the inner layer 300 is joined to the outer layer 100 as indicated by the dotted line 260. The front panel and the back panel may be joined by stitching along sides 150 and 155 in a conventional manner. The inner layer 300 is shorter in length than the outer layer 100 as indicated by the dotted line 170.

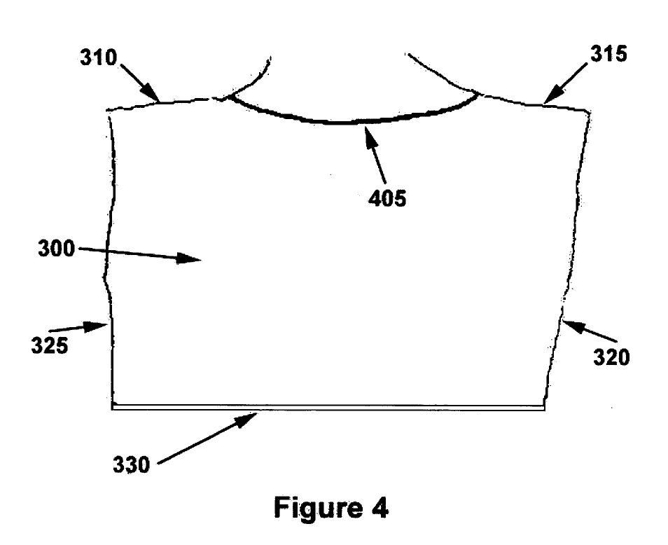

The inner layer 300 of a garment, as shown in FIGS. 3 and 4, in accordance with an exemplary embodiment of the present invention is of a similar shirt design as the outer layer. However, the inner layer is preferably smaller in width below the arm holes to allow for a relatively tight fit against the body and shorter in length, so as to be concealed by the outer layer. The material of the inner layer is preferably a stretchable fabric (e.g., Lycra® by E. I. Du Pont de Nemours and Company), a tight jersey knit, a polyester stretch, a cotton, a blend of the above fabrics, or other suitable fabrics. The material should be chosen with patient comfort in mind, especially for patients with sensitive skin. The inner layer may otherwise follow the same general shirt pattern as the outer layer.

In a preferred embodiment the inner layer 300 is attached to the inside of the outer layer 100. For example, the neckline 305 of the inner layer may be stitched to the neckline 105 of the outer layer. Shoulder seams for a front panel of the inner layer 310 and 315 may be attached to shoulder seams for a front panel of the outer layer 115 and 120. Likewise, shoulder seams for a rear panel of the inner layer may be attached to shoulder seams for a rear panel of the outer layer. Preferably, upon opening releasable closure means 715-730 provided at the shoulders of the outer layer, the shoulders of the outer layer and the shoulders of the inner layer may be opened. Thus, in an exemplary embodiment, the releasable closure means for the outer layer operate as releasable shoulder closure means for the inner layer.

The sides 320 and 325 of the inner layer 300 are joined from the arm holes to the bottom hem 330 in a conventional manner, but independently from the outer layer 100 and sides 150 and 155.

A bottom hem 330 for the inner layer 300 is preferably comprised of an elastic band. For example, a ¾-1″ inch width band may be used, though narrower or wider elastic can also be used. Lingerie or cotton elastic is preferred for comfort. The elastic is preferably smaller than the chest dimension for the size of the garment. For example; a woman's large may measure thirty inches at the chest and twenty-seven inches at the bottom hem. Preferably, the elastic band provides a comfortable but secure fit to prevent the free ends of the catheter lines from falling below the hem under normal conditions, while protecting the catheter port site and lines.

FIG. 4 demonstrates the rear panel of the inner layer 300. As described in FIG. 3, the rear panel of the inner layer 300 remains the same with the exception that neckline 405 has been raised to the same level as the outer layer neckline 205 and is stitched in the same manner as 105 and 305 of the front panels.

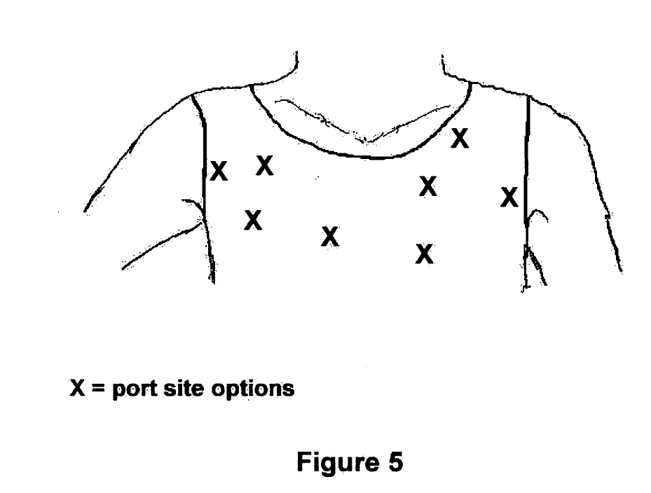

Referring now to FIG. 5, various potential port sites are conceptually illustrated, although not limited to, each by an X. A garment in accordance with an exemplary embodiment of the present invention conceals, and therefore secures, port sites within the boundaries of the garment. Thus, the garment accommodates a wide range of port sites. It does not have openings or pockets that require certain specific port site locations.

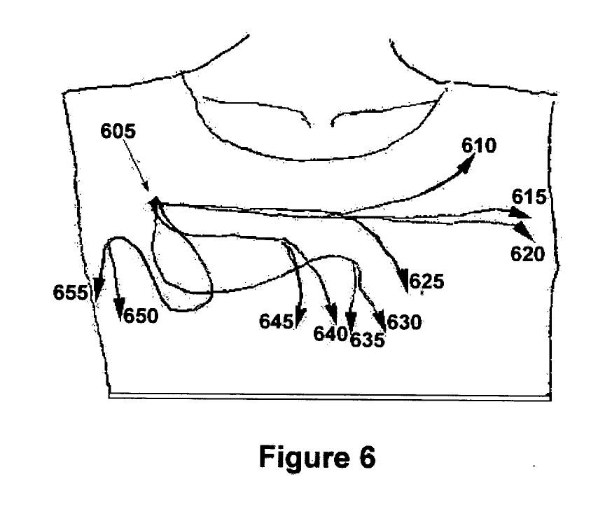

Referring now to FIG. 6, various locations and positioning of the external line portion of a catheter are conceptually illustrated, each by a curved line with an arrow. The external portion of each line starts at a port site 605 and ends with an access port (denoted by an arrow). Though lines with a plurality of lumens or single lumen may be used, double lumen lines are conceptually shown in FIG. 6 to illustrate a range of placements for a given port site. For clarification, lumens 610 and 625 stem from the same catheter line, as do 615 and 620, 630 and 635, 640 and 645, and 650 and 655. Clips, which are commonly used with lines/lumens, are also not shown in FIG. 6. Of course, the port site shown in FIG. 6 is but one example of a port site. Other port sites within the boundaries of the garment may be used without adversely reducing the efficacy of the garment.

A garment in accordance with an exemplary embodiment of the present invention conceals and secures lines in a wide range of positions, as demonstrated but not limited to, 610-655 within the boundaries of the garment. Thus, the garment allows positioning of a line to maximize comfort. By way of example, a patient wishing to sleep on his or her side or stomach may position the line (and particularly the access ports and any clips, both of which can be hard), in a location that enhances comfort. Advantageously, the garment does not depend upon immovable openings or pockets for locating the line.

According to an exemplary embodiment of the invention the inner layer is used with an outer layer. Advantageously, the outer layer helps insulate the inner layer from stresses and strains attributed to movement of a patient's outer clothing. Without an outer layer, movement of outer clothing could possibly cause the inner layer to move against the patient's body and thus cause complications with the port site and catheter lines. The outer layer is designed to reduce that risk allowing the inner layer to do what it is designed to do—protect and secure the port site and line. As described above, the outer layer may be joined to the inner layer at the neckline, but not at the bottom (waist) of the garment. Thus, the bottom of the outer layer may shift independent of the inner layer. Such independence, prevents snagging of the outer garment from imparting stresses to the central line protected by the inner layer. This attachment arrangement of an independently movable outer layer which is not attached to the inner layer at the bottom thus provides an important advantage over conventional single layer garments and garments with separate layers attached at the bottom and top.

Another important aspect of the invention is that the inner layer provides compressive support adequate to comfortably secure lines in a wide range of positions, without exerting excessive compressive pressure. In other words, the support provided by the inner layer of a properly sized garment according to the invention is adequate to secure a wide array of catheter lines and lumens against the wearer's body in a wide variety of positions, without causing discomfort from excessive compression. Preferably, the compressive support provided by a properly sized inner layer is substantially uniform, such that the force exerted against a catheter line at one position is substantially similar to the force exerted at another position (i.e., within the tightness range as described below).

In a preferred implementation, the magnitude of the compressive force provided by a properly sized garment according to an embodiment of the invention is within a tightness range, which is referred to herein as an effective tightness or an effectively tight fit. At the lower end of the range is a compressive force adequate to hold a line against a wearer's body. At the higher end of the range is a compressive force that is a few times (e.g., two to three times) the compressive force at the lower end of the range. Below the lower end of the range, the inner layer would provide inadequate compression to secure a line against a wearer's body. Any fit that does not provide substantial excess space between the garment being worn and an intended wearer is at or above the lower end of the range, so long as it is sufficient to support a line against a wearer in a plurality of positions. Because the exemplary outer layer is not intended to provide compressive support, it provides less than an effectively tight fit. Above the higher end of the range, the inner layer would provide substantially excessive compression to secure a line against a wearer's body and may eventually cause extreme discomfort. Any fit that substantially exceeds compression to secure a line against a wearer's body and causes appreciable discomfort is considered above the higher end of the range. Sports bras and other similar athletic support garments for females fall within this category.

Those skilled in the art will appreciate that a garment in accordance with the present invention when worn by a person for whom the garment size is not intended (e.g., an extra large garment worn by a petite person or child, or a small garment worn by a large person) will not fit the wearer properly. Nevertheless, such a garment is still considered to provide an effective tightness or effectively tight fit if it provides sufficient compressive force to secure a catheter line, but not substantially excessive compression that will cause extreme discomfort when worn by a person for whom the garment size is intended.

Stretch fabrics capable of providing an effective tightness are used for the garment. The stretchable material content in the fabric may vary greatly, depending upon the amount of stretch and support needed, or desired. The fabric for the inner layer should be, but is not limited to being, a two-way stretch interlock knit. By way of example and not limitation, the materials may include blends of spandex with cotton, nylon, polyester, rayon, silk, or other stretchable materials. Spandex is a generic name for a variety of elastic textile fibers made chiefly of polyurethane. Lycra® is one example of a spandex fabric available from E. I. duPont de Nemours & Co., Wilmington, Del. Various weights of fabric can be used for the inner layer and outer layer. For example, light, light/medium, medium, medium/heavy, and heavy weights may be utilized. Different weights are preferred for different purposes. In a preferred implementation an 8 ounce (or no less than 4 ounce) double knit 2-way stretch may be used for the inner layer, and an 8 ounce double knit cotton/spandex blend with 5-10% spandex (or a 4 ounce interlock cotton fabric) may be used for the outer layer. The garment should be constructed with the stretch of the inner layer of the garment oriented across the body of the wearer (i.e., horizontally), not up and down (vertically), such that the circumference of the inner layer is allowed to expand. All of these fabrics and blends can provide an effective tightness, excellent comfort and support. Obviously, a complete array of colors and prints are suitable for both the inner layer and outer layer of the garment.

It is the multi-directional stretch characteristics of the fabric, in conjunction with the specific design, that gives the garment, particularly the inner layer, an ability to conform to wearer's physique and provide an effective tightness. The inner layer conforms to and provides a non-constricting, non-restricting, effectively tight fit. The chest and torso are allowed to expand and contract without appreciable resistance. The garment completely accommodates the line in various positions for comfort without pressing the line or lumens into the wearer's body.

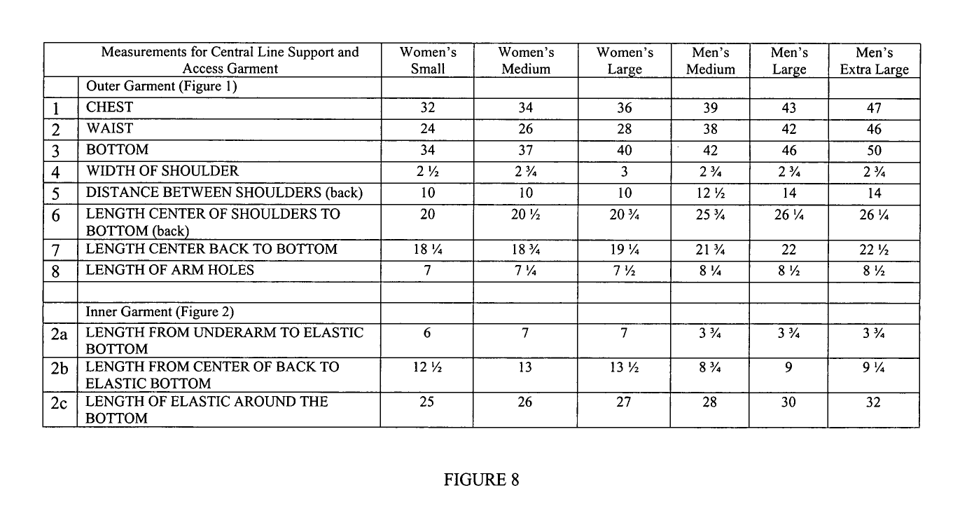

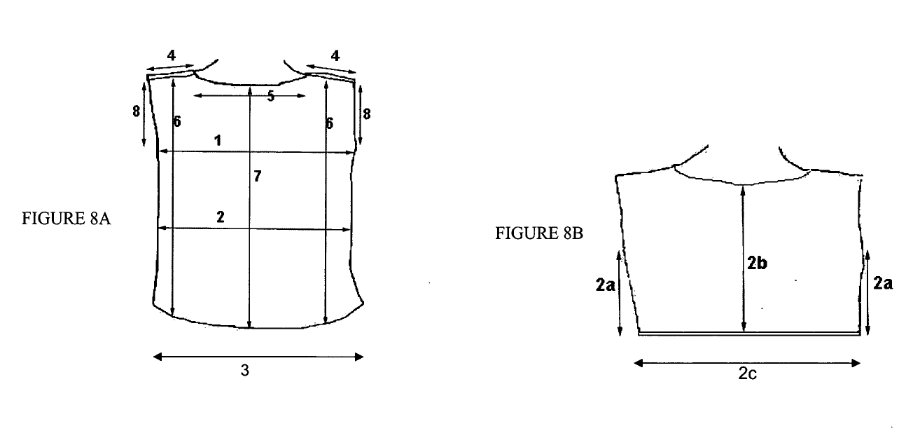

Those skilled in the art will appreciate that certain patterns of stretchable materials will achieve a determined effective tightness when worn by a person of an appropriate size. By way of example and not limitation, the inner layer constructions described in FIGS. 8, 8A and 8B are considered to provide an effective tightness in accordance with the invention, when the materials for the inner and outer layers conform to the preferred material specifications set forth above.

Several aspects of conventional sports bras and other similar athletic support garments for females (referred to collectively herein as “sports bras”) render such garments highly undesirable for use in securing a catheter line against a wearer's body. Most significantly, in sharp contrast to a garment according to the invention, conventional sports bras are designed to exert substantial compressive force to minimize breast motion. A strong elastic material holds the breasts firmly against the wearer's body. While such compressive force is necessary for minimizing breast movement during rigorous activity, it is excessive for supporting a catheter line and leads to great discomfort, pain and swelling when exerted on a regular basis. The discomfort is particularly pronounced when a portion of the wearer's body is swollen and tender, such as soon after implantation of a catheter line. Such compressive force does not constitute an effectively tight fit as contemplated herein.

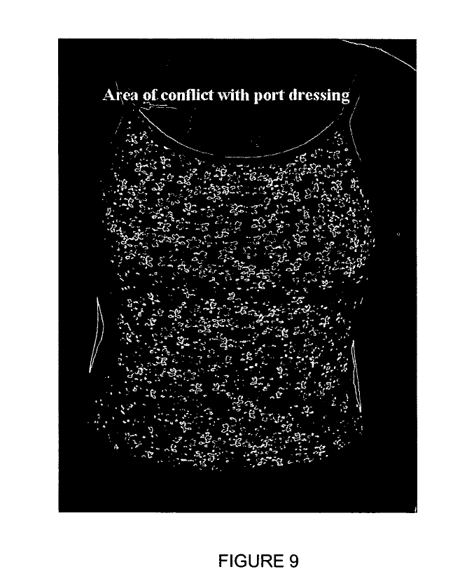

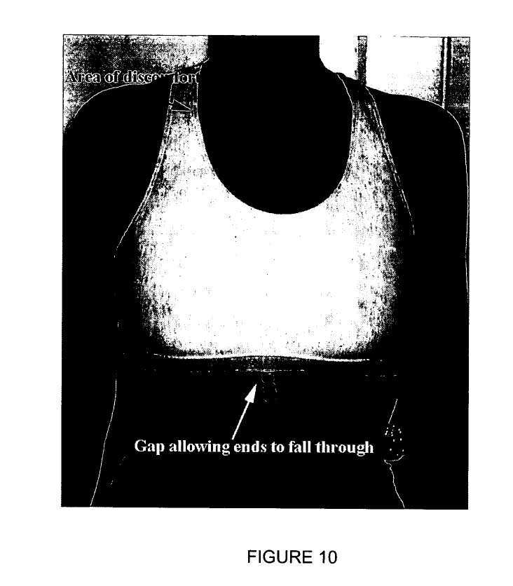

Another aspect of sports bras and certain other garments that render them highly unsuitable for supporting a catheter line is that the straps are often very thin. Garments having thin straps are extremely problematic because the straps cut into the shoulders of the wearer, particularly larger and more robust wearers. Catheters are often placed into a vein in a patient's neck (internal jugular) or sometimes under the collar bone (subclavian) and tunneled under the skin. Conventional thin straps of sports bras tend to cross a wearer's collarbone over the tunneled catheter line. As conceptually shown in FIGS. 9 and 10 illustrate a sports bra and a garment with spaghetti straps and particular areas of conflict or discomfort with a catheter port or line. Concentrated pressure from thin straps may cause discomfort by abrading or occluding a tunneled catheter line that cross a wearer's clavicle.

Advantageously, a garment according to an exemplary embodiment of the invention avoids placing undue stress on the clavicle region by utilizing wide shoulder supports. Although thin straps such as spaghetti straps may be stylish, they are unsuitable for a garment according to the invention. The shoulder portions of a garment according to the invention are preferably approximately 1½ inches or wider, which is several times wider than conventional spaghetti straps and bra straps. This width is considered minimally effective for avoiding stress concentrations that substantially increase discomfort. The shoulder portions may take the form of wide (1½ inch or wider) straps, T-shirt-style sleeves, or any other shoulder form that provides at least 1½ inches of shoulder support material for avoiding severe stress concentrations at the shoulder region. The width of such shoulder supports is referred to herein as an effectively wide shoulder.

Another problem associated with conventional sport bras and other garments is that they are difficult to put on. For instance, sport bras are typically designed to be placed on over the head and require stretching of the elasticized band in order to do so. Other garments may require tying of straps at the shoulders or buckling of straps across the wearer's back. However, patients with implanted catheters frequently experience soreness, tenderness and an extremely limited range of motion, which significantly impairs their ability to put on such garments.

Advantageously, a garment according to the invention is easy to put on. A wearer can easily put on a garment according to an exemplary embodiment of the invention by pulling the garment over their head, in which case the shoulders may be fastened before or after the garment is pulled on. Alternatively, the wearer can step into the garment, pull it up and then fasten the shoulder portions. Because the shoulder portions are releasably joined, such as using hook and loop fasteners, they are easy to close when the garment is in place or before putting the garment on.

The foregoing detailed description of particular preferred embodiments of the present invention, which should be read in conjunction with the accompanying drawings, is not intended to limit the enumerated claims, but to serve as particular examples of the invention. Those skilled in the art should appreciate that they can readily use the concepts and specific embodiments disclosed as bases for modifying or designing other garments carrying out the same purposes of the present invention, and that such garments come within the scope of the invention.

By way of example, and not limitation, garments with various additional pockets, pouches, and accessories come within the scope of the present invention. Also by way of example and not limitation, garments comprised of other materials than those identified above, e.g., breathable, lightweight and sheer materials, as well as synthetic materials or blended materials, whether now known or later developed, may come within the scope of the present invention. Those skilled in the art should also realize that such equivalent garments do not depart from the spirit in scope of the invention as claimed.

Having thus described exemplary embodiments of the invention, what is claimed as new and desired to be secured by Letters Patent is as follows:

1. A central line access and support garment for protecting a catheter line and port site, said garment comprising
an inner layer adapted to provide an effectively tight fit, said inner layer having a bottom and an elastic band at or adjacent to the bottom, said elastic band providing an effectively tight fit, and
an outer layer.

2. A central line access and support garment according to claim 1, wherein the effectively tight fit is substantially uniform.

3. A central line access and support garment according to claim 1, wherein said outer layer is adapted to provide less than an effectively tight fit.

4. A central line access and support garment according to claim 1, wherein
said inner layer includes a top and bottom, and
said outer layer includes a top and bottom, and said inner layer and said outer layer are attached at or near the top of the inner layer and the top of the outer layer.

5. A central line access and support garment according to claim 4, wherein said inner outer layer is adapted to move substantially independent of the inner layer.

6. A central line access and support garment according to claim 1, said garment further comprising a left shoulder section, a right shoulder section and means for releasable closure of the left and right shoulder sections.

7. A central line access and support garment according to claim 6, wherein said left shoulder section is adapted to provide an effectively wide shoulder, and said right shoulder section is adapted to provide an effectively wide shoulder.

8. A garment according to claim 1, said inner layer being comprised of at least one material from the group consisting of cotton, stretchable synthetic material, jersey knit and polyester.

9. A garment according to claim 1, said outer layer being comprised of at least one material from the group consisting of cotton, stretchable synthetic material, jersey knit and polyester.

10. A central line access and support garment for protecting a catheter line and port site, said garment comprising
an inner layer adapted to provide an effectively tight fit, and
an outer layer.

11. A central line access and support garment according to claim 10, wherein the effectively tight fit is substantially uniform.

12. A central line access and support garment according to claim 11, wherein said outer layer is adapted to provide less than an effectively tight fit.

13. A central line access and support garment according to claim 12, wherein
said inner layer includes a top and bottom, and
said outer layer includes a top and bottom, and said inner layer and said outer layer are attached at or near the top of the inner layer and the top of the outer layer.

14. A central line access and support garment according to claim 13, wherein said inner outer layer is adapted to move substantially independent of the inner layer.

15. A central line access and support garment according to claim 14, said garment further comprising a left shoulder section, a right shoulder section and means for releasable closure of the left and right shoulder sections.

16. A central line access and support garment according to claim 15, wherein said left shoulder section is adapted to provide an effectively wide shoulder, and said right shoulder section is adapted to provide an effectively wide shoulder.

17. A garment according to claim 16, said inner layer being comprised of at least one material from the group consisting of cotton, stretchable synthetic material, jersey knit and polyester.

18. A garment according to claim 17, said outer layer being comprised of at least one material from the group consisting of cotton, stretchable synthetic material, jersey knit and polyester.

19. A method for protecting a central catheter implanted in a patient, said method comprising steps of:
placing a garment according to claim 1 on the patient, and
positioning between the inner layer of the garment and the patient's body a portion of the central catheter that is outside the patient's body.

20. A method for accessing a central line catheter protected using a garment according to claim 6, said method for accessing the central line comprising steps of:
releasing a releasable closure means for a shoulder section of the outer garment,
folding the shoulder section downward to expose at least a portion of the patient's body that was previously covered by the garment, and
manually directing the portion of the central line that is outside the patient's body to the exposed portion of the patient's body.
